(19) United States
(12) Patent Application Publication
(10) Pub. No.: US 2006/0275821 A1
(43) Pub. Date: Dec. 7, 2006

(54) Method for isolating subpopulations of proteins that engage in protein-protein interactions

(76) Inventors: Thomas J Nelson, Rockville, MD (US); Daniel L. Alkon, Bethesda, MD (US)

Correspondence Address: TIM HERLIHY, 706 STAG HEAD ROAD, TOWSON, MD, 21286, US

(21) Appl. No.: 10/519,109

(22) PCT Filed: Aug. 14, 2002
(86) PCT No.: PCT/US02/25845

§ 371 Date: Jul. 26, 2006

(51) Int. Cl. G01N 33/53; C07K 1/12; C07K 14/47

(52) U.S. Cl. 435/7.1; 530/412; 530/409

(57) ABSTRACT

The invention provides a method for isolating and identifying proteins participating in protein-protein interactions in a complex mixture. The method uses a chemically reactive supporting matrix to isolate proteins that in turn non-covalently bind other proteins. The supporting matrix is isolated, and the non-covalently bound proteins are subsequently released for analysis. Because the proteins are accessible to chemical manipulation at both the binding and release steps, identification of the non-covalently bound proteins yields information on specific classes of interacting proteins, such as calcium-dependent or substrate-dependent protein interactions. This permits selection of a subpopulation of proteins from a complex mixture on the basis of specified interaction criteria. The method has the advantage of screening the entire proteome simultaneously, unlike two-hybrid systems or phage display methods which can only detect proteins binding to a single bait protein at a time. The method is applicable to the study of protein-protein interactions in biopsy and autopsy specimens, to the study of protein-protein interactions in the presence of signalling molecules, pharmacological agents or toxins, and for comparison of diseased and normal tissues or cancerous and untransformed cells.

### FIELD OF THE INVENTION

The invention relates to the study of protein-protein interactions and is expected to be useful in the fields of biochemical signal transduction, proteomics, drug discovery, toxicology, and diagnostics.

### BACKGROUND OF THE INVENTION

Protein-protein interactions underlie a vast number of physiological processes. Cellular processes such as neuronal signaling, cell development, growth, and replication all depend on a complex network of protein-protein and protein-small molecule interactions in the cell. These interactions may be categorized as constitutive interactions, such as between subunits of hemoglobin, and signal-dependent interactions, such as those between the subunits of cAMP-dependent protein kinase or the subunits of GTP-binding proteins. The complexity of the task of investigating these interactions is evident from the potential number of protein interactions: comprehensively screening binary interactions among 15,000 proteins would require testing over 2×10⁸ pairwise combinations of proteins. This complexity means that conventional biochemical methods are of limited use. Despite intensive research, there is still no satisfactory method for systematically studying protein interactions in mammalian cells or other complex mixtures of proteins.

A number of techniques have been used to study individual protein-protein interactions, including protein cross-linking [1, 2, 3], green fluorescent protein [4, 5], phage display [6, 7], the two-hybrid system [8], protein arrays [9], fiber optic evanescent wave sensors [10, 11], chromatographic techniques [12], and fluorescence resonance energy transfer [13, 14]. However, these methods are generally useful for screening only one bait protein at a time. For a brief review, see [15].

Extensions of these methods to the identification of in vivo protein-protein interactions on a moderately large scale have been reported. One approach involves the generation of fusion proteins of proteins of interest with “tandem affinity purification” (TAP) tags [16]. The fusion proteins, together with any proteins with which they form complexes, are isolated by a two-step affinity purification process based upon the TAP affinity tags, and the associated proteins are identified by a combination of gel electrophoresis and mass spectrometry. The other reported method employs a very similar strategy, using epitope tags and a one-step affinity purification process [17]. Both methods were rendered “high-throughput” by a brute force approach, which involved individually processing over 1,700 genes and over 1,000 individual yeast expression clones in the former case and 725 genes in the latter.

Rappsilber et al. [18] have also carried out affinity purification of protein complexes, followed by cross-linking of the associated proteins

The above methods are limited by the need to prepare and express individual fusion proteins, and the reliance on recombinant expression hosts severely limits the variety of cell types that can be studied. The TAP tagging method also exhibits a bias against proteins below 15 kDa, and both TAP and epitope tags may interfere with normal protein-protein interactions.

With the aid of high-throughput robotics, the two-hybrid system has also been adapted for proteomic screening. In these experiments [19, 20], a Gal4 library of 6000 yeast proteins was partitioned into separate wells of 96-well plates, and each well was screened against an activation domain library, yielding as many as 4,549 possible interactions among yeast proteins.

Unfortunately, this technique is infeasible for screening mammalian proteins because it would result in a large proportion of non-specific interactions. Mammalian proteins are normally expressed in a variety of compartmentalized subcellular organelles and in specific cell types, and are extensively post-translationally modified in a tissue-specific manner. There is no technology available for performing a two-hybrid analysis on a tissue sample. Thus, natural protein interactions associated with physiological processes such as learning or Alzheimer's disease, or interactions resulting from signaling, cannot be studied with this technique.

### BRIEF DESCRIPTION OF THE INVENTION

The invention provides a method for screening of both constitutive and signal-mediated protein-protein interactions. The method of the invention has several advantages:

(1) It is a protein-based technique, and does not require cloning but uses the native proteins in their native, folded state which are properly post-translationally modified;

(2) The interactions are accessible to chemical manipulation, permitting interesting subpopulations of protein-protein interactions, such as calcium-dependent protein-protein interactions, to be easily studied;

(3) Perturbations of protein-protein interactions by pharmacological agents or toxins, or differences between cancerous and untransformed cells, can also be screened; and

(4) If an appropriate control group is used, only naturally-occurring protein interactions are observed. Non-physiological interactions are eliminated from analysis because non-physiological interactions are identical in both groups.

The method uses an activated solid support to isolate proteins that are non-covalently bound to other proteins. The support is preferably a gel, and is more preferably agarose. The support is activated by the presence of chemically-reactive functional groups that are capable of covalently binding proteins. Cyanogen bromide-activated Sepharose™ is a preferred support. Because the interacting proteins are subject to experimental environmental manipulation, mass spectrometric identification of the proteins can yield information on specific classes of interacting proteins, such as calcium-dependent or substrate-dependent protein-protein interactions. This permits the selection and isolation of a subpopulation of proteins from a complex mixture on the basis of specified interaction criteria.

The method enables the simultaneous screening of an entire proteome, unlike two-hybrid systems or phage display which can only detect proteins binding to a single bait protein at a time. Since only naturally-occurring interactions of proteins in their native state are observed, this method will have wide applicability to studies of protein interactions in tissue samples and autopsy specimens, for screening for perturbations of protein-protein interactions by signaling molecules, pharmacological agents or toxins, and screening for differences between cancerous and untransformed cells.

In its broadest aspect, the invention provides a method of isolating, from a mixture of proteins, a subpopulation consisting essentially of proteins that engage in protein-protein interactions. The method comprises the steps of (a) contacting the protein mixture with a chemically reactive support, under conditions that permit both covalent binding of proteins to the support and protein-protein interactions; (b) permitting proteins in the mixture to become covalently bound to the support; (c) separating the support from any proteins not bound to it; (d) subjecting the support to conditions that disrupt protein-protein interactions; and finally (e) separating the support from any proteins not bound to it. The proteins released in step (e) are those proteins that non-covalently bound other proteins under the conditions of step (a).

The chemically reactive support may contain any chemically reactive functional group capable of covalently binding proteins in an aqueous environment. Preferred chemically reactive moieties include but are not limited to cyanate, isocyanate, isothiocyanate, activated carboxyl, activated sulfonyl, aldehyde, epoxide, and thiol groups. Particularly preferred is the cyanate group.

The support may be any matrix that is physically separable from the reaction mixtures. The support is preferably in the form of particles or beads. Preferably the support comprises an optionally cross-linked polymer or gel. Preferred support materials include but are not limited to polystyrene, agar, agarose, polyacrylamide, dextran, hydroxylated vinyl polymers, and carboxylated vinyl polymers. A particularly preferred support comprises agarose, for example the varieties of cross-linked agarose sold under the trade name Sepharose™.

The methods of this invention are also useful in the analysis of protein interactions with protein microarrays. In conventional microarray applications, an entire proteome is applied to a microarray of immobilized proteins to investigate protein-protein interactions. [21, 22] However, any specifically-binding proteins of interest are in competition with an enormous excess of proteins that do not participate in specific protein interactions. These excess proteins may be adsorbed nonspecifically onto the microarray and/or compete for binding sites by virtue of their greater concentration in the mixture. Also, the relatively large mass of protein requires a proportionally large volume of solubilizing buffer, which reduces the concentration of proteins of interest. Through concentration effects, fluorescence quenching, competition, and dilution, the presence of a large quantity of irrelevant proteins can greatly reduce the signal-to-noise ratio obtained from a protein microarray.

The present invention reduces these problems by pre-selecting a subpopulation of proteins on the basis of their ability to interact with other protein targets. This subpopulation contains precisely those proteins that are likely to bind specifically to a protein microarray. The method of the invention will reduce the total number of distinct proteins in a proteome by at least 75% prior to application to a protein microarray. The proteins that are eliminated are those that do not participate in specific protein-protein interactions, including many that have the potential to be non-specifically adsorbed or trapped on the array.

The retained proteins may be labeled if desired (e.g., with biotin or an appropriate fluorescent dye) and applied to a protein microarray in the same manner as is currently done in existing applications. In so doing, the potential noise on a protein microarray from the unwanted proteins is reduced considerably, resulting in a substantial improvement in the quality of the results obtainable from protein microarrays. Another application of the method to microarrays results from the ability of the method to easily isolate protein subpopulations with specific, desired biochemical properties. For example, the practitioner may use the method to select for proteins whose interactions depend on the presence of calcium, cAMP, a specific DNA sequence, or a pharmacological agent such as rapamycin. By manipulating the wash conditions, proteins with relatively low or relatively high affinity may be selected for. When the method of the invention is employed to pre-select for proteins of interest, the microarray is more likely to successfully identify the proteins involved in an interaction. The method permits researchers to perform tests that would otherwise require construction of specialized microarrays or other expensive or indirect methods to achieve similar results.

Thus, in a method for analyzing a mixture of proteins which comprises contacting the mixture with an array of immobilized proteins, the method of the invention provides an improvement which consists of isolating, from the mixture of proteins, a subpopulation consisting essentially of proteins that engage in protein-protein interactions, before the subpopulation is subsequently contacted with the array.

The method of the present invention has been described in a publication [23].

### BRIEF DESCRIPTION OF THE FIGURES

FIG. 1A shows the six possible outcomes of binding a bimolecular protein complex (AB) to Sepharose™ support particles.

FIG. 1B illustrates the elution of non-covalently associated proteins from the Sepharose™ particles of FIG. 1A.

FIG. 2 is a Western blot of an SDS-PAGE gel, comparing the calmodulin-binding proteins isolated using the CNBr-Sepharose™ method (left) to those isolated by calmodulin-affinity chromatography (right).

FIG. 3 Coomassie blue-stained 2-dimensional gel of rat brain extract subjected to enrichment of Ca2+-dependent protein-protein interactions by the method of the invention. Fewer than 200 spots are visible on this gel, indicating a selected subpopulation of proteins. Of the 23 largest spots, the 12 indicated spots were identified by mass spectrometry.

FIG. 4 is a 2-dimensional gel of rat brain proteins released from CNBr-Sepharose with 8M urea.

FIG. 5 shows the calmodulin spot in a 2-dimensional gel after selection for Ca²⁺ dependent protein-protein interactions (left), compared to the calmodulin spot in a 2-D gel without selection (right). Identical amounts of total protein (100 μg) were applied to each gel. The calmodulin spot is enriched approximately 50-fold.

FIG. 6 shows a potential application of the method in the investigation of learning-dependent changes in protein interactions. The panels are corresponding regions of 2-D polyacrylamide gels of proteins from hippocampal extracts of rats trained (left panel) and untrained (right panel) in a water maze. Two proteins (center of left panel) are candidates for learning-specific alterations in protein-protein interactions.

### DETAILED DESCRIPTION OF THE INVENTION

Abbreviations:

CHAPS: 3-[(3-cholamidopropyl)dimethylammonio]-1-propane sulfonate

EGTA: Ethylene glycol bis-(2-aminoethyl ether)-N,N,N′,N′-tetraacetic acid

MARCKS: Myristoylated Alanine-Rich C Kinase Substrate

Non-physiologically relevant interactions are a significant potential problem in many studies of protein-protein interactions, including the two-hybrid system. In the present method, when a tissue sample is homogenized, in addition to protein interactions caused by the treatment, nonspecific protein interactions will also occur between proteins that normally are not in contact with each other. For example, interactions between membrane and nuclear proteins, or between astrocyte and neuronal proteins could occur. To prevent this from producing false interactions, it is necessary to use both a treatment and control sample and consider only differences between the two samples as representing potentially significant interactions. Since any nonspecific or nonphysiological protein interactions occurring after homogenization of the cells would be identical in both the control and treatment groups, it is easily possible to distinguish normal, physiologically-relevant protein-protein interactions from artifactual ones. Nonspecific interactions would be identical in both the control and treated groups, while changes in binding that occurred in vivo caused by the treatment would be readily detectable.

As an example of such a differential analysis, the method could be used to study protein interactions that may occur after associative learning in the rat water maze task [24, 25]. This experiment uses a trained group, which swims in a tank of water containing a concealed platform, and an untrained control group, which is allowed to swim in a tank with no platform. Interacting proteins from hippocampal extracts of each group would then be isolated separately using the CNBr-Sepharose™ method and analyzed on 2-dimensional polyacrylamide gels. Any differences in protein interactions produced by learning would be reflected as spots for which the intensity in the trained group differs from the intensity of the corresponding protein spot in the control groups. FIG. 6 demonstrates the results from such an experiment, with two proteins possibly exhibiting a learning-specific increase in protein interactions (upper center in left panel). Once these proteins are identified by mass spectrometry or other means, their binding partners can be easily identified. This could provide a useful means of identifying new signaling pathways relevant to physiological processes.

Other examples of the use of this differential analysis would be testing for protein interaction differences between normal and cancer cells, and determining the in vivo effects of a pharmacological agent or toxin. Any differences between the two groups cannot result from artifactual interactions occurring during sample processing, but represent differences in in vivo protein interactions produced by the treatment.

When isolating in vitro interacting proteins, such as the set of proteins that undergo calcium-dependent interactions or the set whose interactions depend on the presence of some pharmacological agent, the calcium or pharmacological agent being tested would be added to the buffer at each step in sufficient concentration to ensure tight binding of all relevant proteins. During the elution step, instead of 8M urea, the proteins would be eluted by washing the chromatography column with a buffer identical in all respects except that the pharmacological agent is omitted. The ionic strength and pH of the two buffers should be identical to avoid eluting any proteins by virtue of a change in pH or ion concentration. In this case, it is not strictly necessary to compare two separate samples, because the protein interactions of interest are those occurring in the test tube.

Although this method can give as much as 50-fold enrichment of interacting proteins, it is possible that some noninteracting proteins could also be detected if their affinity for Sepharose™ is higher than the affinity of the proteins for each other. Although protein adsorption to Sepharose™ is possible [26], the effects are generally small, and would be eliminated by adding a control group as described above. Non-specific binding to Sepharose™ was not observed in the experiments described herein.

The data produced using this method will consist of raw information concerning proteins that may interact with some other, as yet unidentified protein or proteins. Although this is valuable information in itself, and reduces the problem space by several orders of magnitude, it is still necessary to validate the putative protein interactions. Once the target proteins are identified, their binding partners can be easily found using conventional techniques such as affinity chromatography [27] or two-hybrid analysis. To provide a complete understanding of the interaction, it also necessary to confirm that the putative interaction occurs using some alternative method. Confirmation of the observed change is, of course, also necessary in other screening methods such as DNA microarrays, phage display, or the two-hybrid system. Since the present method only measures the total quantity of protein in an interacting state, validation of the interaction is also needed to determine the biochemical basis for the increased levels of interaction, which could be produced by greater affinity of the target protein, greater abundance, or even, in unusual cases, induction of an activator or reduced levels of some inhibitor of the interaction.

This technique could also be modified by adding a cross-linking step after the initial wash, and substituting thiol-Sepharose™ for CNBr-Sepharose™. This would permit the pair of interacting proteins to be separated by cleavage of the disulfide bond linking the protein to Sepharose™, allowing the crosslinked protein pair to be separated and identified as a single unit.

Besides calcium-dependent protein interactions, numerous examples exist of protein-protein interactions mediated by GTP, cAMP, protein phosphorylation, enzyme substrates, or other biochemical phenomena. The present method could be used to investigate these categories of protein interactions, for example by comparing patterns produced in the presence or absence of a protein phosphatase or nucleotide phosphohydrolase.

The new method has the advantage of screening the entire proteome simultaneously, unlike other methods which can only detect proteins binding to a single bait protein at a time. In addition, the method does not require cloning but isolates naturally-occurring interactions between proteins in their native, folded state that are properly post-translationally modified. The proteins are also accessible to chemical manipulation, permitting selection of a subpopulation of proteins from a complex mixture on the basis of specified interaction criteria. The method would be useful not only for studying protein-protein interactions, but also for identifying the site of action of low-molecular-weight compounds such as xenobiotics or pharmacological agents. Previously, determining whether a xenobiotic affected protein-protein interactions was a daunting task unless one of the target proteins was known. With the current method, the entire proteome can be rapidly screened to identify those proteins whose interactions are affected by a molecule of interest, yielding specific targets for further investigation.

Addition of bifunctional crosslinking reagents to complex protein extracts results in intractable mixtures of often-insoluble aggregates that contain multiple proteins. This is presumably caused by the small molecular size of the crosslinking reagent, which allows it to bind to both interacting partners at multiple locations. A cross-linking agent also permits ordinarily unassociated proteins to bind to one another at random. In the method of the invention, these problems are eliminated because a chemically reactive support is used instead of a crosslinking reagent.

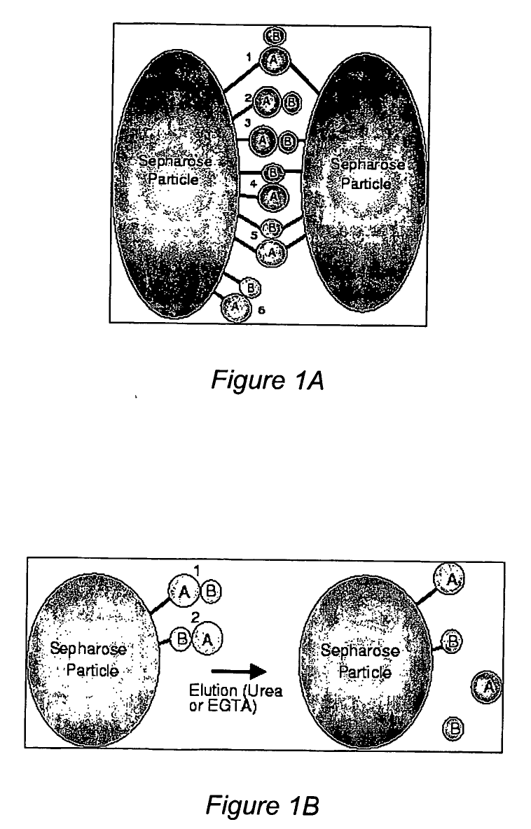

The covalent binding of a pair of interacting proteins A and B to a chemically reactive support could produce six possible categories of outcomes, depending on whether one or both proteins bind to the support. A schematic drawing of a particular embodiment, where the support is Sepharose™ particles, is shown in FIG. 1A. The six possible outcomes, as illustrated, are:

1. Protein A bound to both particles and Protein B free,

2. Protein A bound to a particle and Protein B free,

3. Protein A and protein B bound to different particles,

4. Protein A bound to one particle and protein B bound to both particles,

5. Protein A and B both bound to both particles, and

6. Protein A and B both bound to one particle.

Each line in the figure represents one or more bonds, and the two Sepharose™ particles are assumed to be interchangeable. Three additional outcomes are possible in which A and B are switched. Due to the relatively large size of the particle, outcomes in which the protein complex is bound to different particles (1, 3, 4, and 5 in FIG. 1A) are eliminated because mechanical stress overcomes the chemical bond. Outcomes in which both partners are bound to the same particle (6) should be relatively rare so long as the density of activated groups on the particle is not too high. This leaves only outcome (2), in which one protein is covalently bound to the particle and the other is non-covalently associated with it.

To select non-covalently interacting proteins, the protein mixture is reacted with cyanogen bromide-activated Sepharose™ in such a way that 50% of the proteins are covalently bound. The remaining proteins are either washed away or retained on the Sepharose™ by interacting noncovalently with the covalently-bound proteins. After washing in appropriate buffer, the principal components would be proteins bound at a single site (outcome 2), in which one partner is covalently attached to a particle and its noncovalently attached interacting partner is retained by virtue of its affinity to the bound protein. The noncovalently attached protein is then eluted by washing in 8M urea (FIG. 1B). Alternatively, the elution buffer can be modified to examine specific types of protein-protein interactions, such as substrate-dependent or calcium-dependent interactions. The eluted proteins are analyzed by an appropriate method, such as 2-D gel electrophoresis or capillary LC-MS [28, 29].

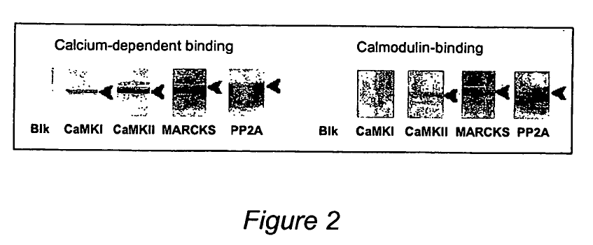

The feasibility of the method as a screening technique was demonstrated by the ability of the method to detect known calcium-dependent protein-protein interactions involving calmodulin. Rat brain homogenate CHAPS extract was bound to CNBr-activated Sepharose™, the Sepharose™ was washed with Tris-acetate buffer, and the interacting proteins were eluted with EGTA. EGTA was used at exactly half the concentration of Tris-acetate, so that no change in the concentration of carboxyl or amino groups, which might elute proteins by virtue of a change in ionic strength, would occur upon the transition to EGTA. Eluted proteins were concentrated, desalted, and separated by 1-dimensional SDS-polyacrylamide gel electrophoresis, blotted onto nitrocellulose, and stained using antibody against calmodulin dependent kinase I, calmodulin dependent kinase II, MARCKS, or protein phosphatase 2A. The Western blot analysis (FIG. 2, left lanes) showed that all four calmodulin-binding proteins tested (CaM kinase I and II, MARCKS, and protein phosphatase 2A) were detectable with the method.

A separate sample of brain homogenate extract was bound to a conventionally-prepared calmodulin affinity column, eluted with EGTA, and the eluted proteins analyzed by Western blotting as described above. The results (FIG. 2, right lanes) were comparable, indicating that the method of the invention is capable of isolating Ca²⁺-dependent calmodulin-binding proteins. The blank (“blk” lane) was from a sample in which brain extract was loaded onto Sepharose™ rendered inert by reacting with Tris-HCl.

By way of example, the use of the method of the invention to investigate calcium-dependent protein interactions will be described. The method was applied to a rat brain extract to select proteins exhibiting calcium-dependent protein interactions. Of 12 proteins identified by mass spectrometry, 8 were either known calcium-binding proteins or proteins with known calcium-dependent protein interactions, demonstrating that the method is capable of enriching a subpopulation of proteins from a very complex mixture on the basis of a specific class of protein interactions.

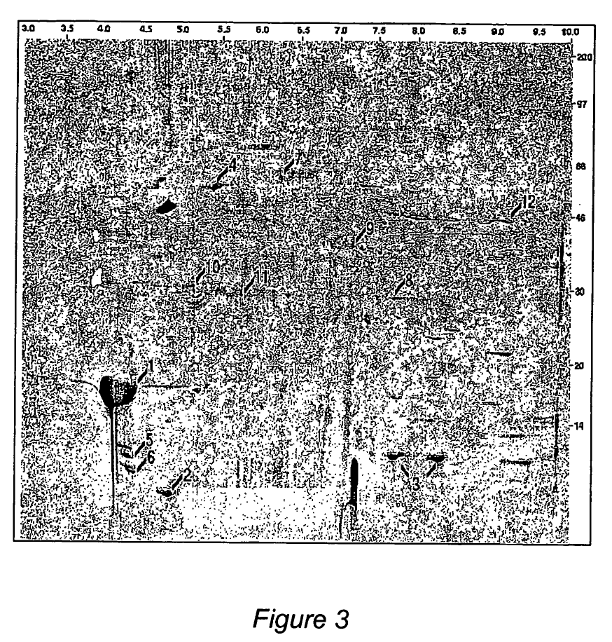

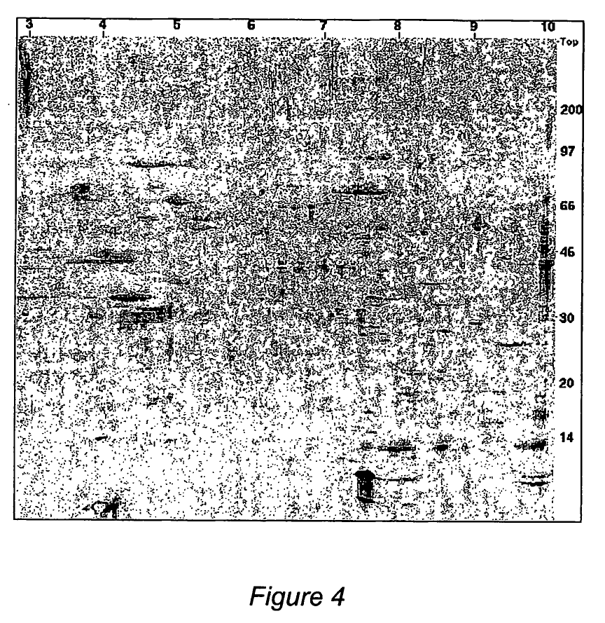

To study the specificity of the method, a sample of rat brain extract was bound to CNBr-Sepharose™ as before, the EGTA-eluted proteins were separated by 2-dimensional polyacrylamide gel electrophoresis. FIG. 3 shows a Coomassie-stained gel from this experiment. The total number of measurable spots (approx. 172) was smaller than the 300-400 spots visible when all interacting proteins were eluted with 8M urea (FIG. 4), and much smaller than the 1000-1200 spots routinely visible from unselected extract (not shown). Twenty-three of the more intense spots detected on the 2-D gel were subjected to digestion with trypsin, and the resulting peptides analyzed by LC-MS/MS. Matching the LC-MS/MS data with the peptide and fragment masses from sequences in the protein database resulted in positive identification for 12 of the 23 proteins analyzed. Of the 12 identified proteins, 8 are proteins known to either bind calcium or interact with other proteins in a calcium-dependent manner (Table 1).

Attempts to identify the remaining 11 large spots in FIG. 3, including the large spots at 10 kDa, pI 7.3 and 52 kDa, pI 4.8, were unsuccessful. Although numerous peptides were obtained, analysis of the mass spectrometric data did not produce a match with any protein in the database.

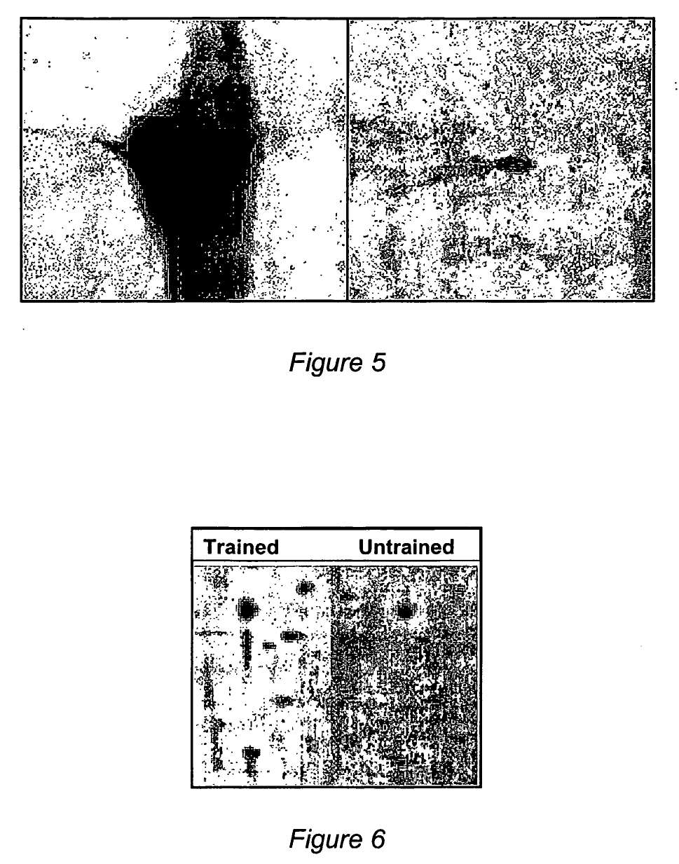

The most abundant protein spot on the 2-dimensional gel (Spot #1) was identified as the calcium-binding protein calmodulin (Table 1 and FIG. 3). This spot is also detectable in crude extract, but is a relatively minor component (FIG. 5). Most of the remaining identifiable spots, including ATP synthase, mitochondrial ATPase inhibitor, and heterogeneous nuclear ribonucleoprotein A2 (hnRNP A2), are also either known calcium-binding proteins or proteins that interact directly with calcium-binding proteins [30, 31, 32, 33, 34]. Peptides from S-100, another calcium-binding protein that undergoes numerous calcium-dependent protein interactions [35], were also detected. Although the M and pI were identical with S-100, because of the small number of observed peptides in the digestion, the mass spectrometric identification did not reach statistical significance.

Table I summarizes the proteins identified by mass spectrometry. With the exception of hemoglobin, citrate synthase, and carbonic anhydrase, all the proteins identified were either known calcium-binding proteins or were proteins with well-characterized calcium-dependent interactions. For example, tropomyosin is associated with the well-known actin-troponin-myosin complex. Ca²⁺ binding to troponin enables troponin to bind tropomyosin and shift it from myosin's binding sites on the actin proteins. Without the presence of Ca²⁺, troponin is no longer able to bind to tropomyosin, tropomyosin again blocks myosin's binding sites on the actin proteins. Tropomyosin also binds to the calcium-binding protein calcyclin [36]. Similarly, Rho GDP dissociation inhibitor strongly binds to the low-MW GTP-binding protein rho, which participates with the calcium binding protein cadherin in reorganization of actin cytoskeleton [37]. Calponin is also a substrate of rho-kinase [38].

TABLE 1
Proteins with calcium-dependent protein interactions

| Spot | | | % Cov- | | |
|---|---|---|---|---|---|
| no. | Mᵣ | pI | erage | Identification | Category |
| 1 | 17,420 | 4.25 | 37 | Calmodulin | Calcium-binding |
| 2 | 52,480 | 4.86 | | α2-mannosidase | Calcium-binding |
| 3 | 5,370 | 4.93 | 21 | S100 beta chain | Calcium-binding |
| 4 | 10,100 | 7.66 | 63 | Hemoglobin | — |
| | | | | alpha 1 | |
| 6 | 60,220 | 5.52 | 48 | ATP synthase | Calcium-binding |
| 11 | 9,770 | 4.42 | 9 | ATPase inhibitor | Calcium-binding |
| 12 | 8,750 | 4.45 | 18 | ATPase inhibitor | Calcium-binding |
| 17 | 61,660 | 6.28 | 12 | Dihydropyrimidase | — |
| | | | | related | |
| 26 | 26,980 | 7.67 | 26 | Carbonic | — |
| | | | | anhydrase 2 | |
| 28 | 36,470 | 8.18 | 2 | heteronuclear | Calmodulin- |
| | | | | RNP A2 | binding |
| 31 | 31,330 | 5.23 | 28 | tropomyosin | Calcyclin-binding |
| 32 | 30,480 | 5.80 | 41 | Rho GDI-1 | Binds Cadherin |
| | | | | | via Rho |
| 35 | 44,510 | 9.061 | 6 | citrate synthase | — |

In the above example, over 30% of the total protein observed was calmodulin, a calcium-binding protein that binds to numerous other proteins in a calcium-dependent manner [39]. Four other proteins (ATP synthase, two forms of ATPase inhibitor, and S100) are also known calcium-binding proteins, while three (tropomyosin, Rho GDP dissociation inhibitor, and heterogeneous nuclear ribonucleoprotein A2 (hnRNP A2), are known to be intimately associated with calcium-binding proteins. It should be noted that the method of the invention is not merely a way to detect calcium-binding proteins. Rather, the method specifically detects the subset of proteins that bind to some other protein in a calcium-dependent manner. This will include some calcium-binding proteins, but it will also include their targets, such as calcium-dependent kinases and signaling proteins (such as rho and rab) which interact with calcium-binding proteins in a calcium-dependent manner.

Three unexpected proteins, hemoglobin, carbonic anhydrase 2, and citrate synthase, were also detected. Although hemoglobin binding to other hemoglobin subunits depends on Fe²⁺ and O₂, it is not known to bind calcium; however, hemoglobin can bind to reticulocyte membranes in the presence of calcium [40], suggesting that it may be a partner for a calcium-binding membrane-bound protein. Similarly, it is possible that citrate synthase and carbonic anhydrase can associate with as-yet uncharacterized proteins in the presence of calcium.

The method described here should be useful for investigating protein-protein interactions in mammalian tissues. For example, it has been suggested that Alzheimer's disease and other neurodegenerative disorders are triggered by pathological protein-protein interactions [41, 42]. Similarly, cell signaling, synaptic plasticity, learning, and development are dependent on a complex network of protein-protein interactions. This method is expected to be useful in isolating macromolecular protein complexes as part of any program of proteomic screening to identify relevant protein-protein interactions for further study.

### Experimental

Titration of CNBr Sepharose™: Cyanogen bromide activated Sepharose™ 4B (Pharmacia) was rehydrated and washed 3 times with water before use. CNBr was titrated with rat brain extract by incubating a fixed quantity of extract at room temperature with varying amounts of CNBr Sepharose™. After 1 hr, samples were centrifuged and the unbound protein was measured using a dye-binding assay[43] and the quantity of CNBr Sepharose™ to reduce the protein concentration by 50% was calculated.

Isolation of interacting proteins: One rat brain was homogenized by sonication in 10 mM NaHCO₃, pH 7.7 containing 5% CHAPS, 0.1 mM phenylmethylsulfonyl chloride, and 1 mM CaCl₂, and centrifuged at 100,000 g for 20 min. A quantity of rehydrated CNBr Sepharose™ sufficient to bind 50% of the protein was added, and the sample was shaken at room temperature for 1 hr. Tris acetate was then added to 0.1M to block unreacted CNBr and incubation was continued for another 30 min. The mixture was transferred to a small chromatography column and washed extensively with 100 mM Tris acetate containing 1 mM CaCl₂. When the A₂₈₀ of the eluate reached zero, the proteins retained by calcium-dependent interactions were eluted with 50 mM EGTA, desalted and concentrated in a Centricon-3 ultrafiltration device, mixed 1:1 with IEF sample buffer (8.5 M urea, 2 M thiourea, 0.4% CHAPS, 0.5% IPG buffer (Amersham), and 0.01% bromphenol blue), and applied to an Immobiline pH 3-10 polyacrylamide isoelectric focusing strip that had been rehydrated with the same solution, and subjected to flatbed 2-dimensional polyacrylamide gel electrophoresis (ExcelGel 12-14).

The gel was stained with Coomassie Blue, and the 23 largest visible spots were excised and subjected to tryptic digestion and LC-MS/MS analysis. Of these, 12 were identified by the SEQUEST and Mascot software as described below.

Affinity chromatograph: Rat brain extract was incubated for 15 min at room temperature with 2 cm3 of calmodulin-Sepharose™ 4B in 50 mM NaHCO₃, pH 7.7 and 1 mM CaCl₂. The mixture was transferred to a column and washed with 100 mM Tris-HCl containing 1 mM CaCl₂ until A₂₈₀ became undetectable. The calmodulin-binding proteins were eluted with 50 mM EGTA, desalted and concentrated in a Centricon-3 ultrafiltration device, separated by electrophoresis in a 4-20% SDS polyacrylamide gel, and blotted onto nitrocellulose membranes.

Western blot analysis: Samples were analyzed by electrophoresis on a 4-20% acrylamide gradient SDS gel, followed by blotting onto nitrocellulose, probed with antibody, and visualized with nitro-blue tetrazolium and 5-bromo-4-chloro-3-indolyl phosphate.

Mass spectrometry: Stained protein spots were excised from the 2-dimensional gel and digested with trypsin, using the in-gel method described by the Association of Biomolecular Resource Facilities [44]. Digestion with trypsin was carried out overnight at 37° C., and peptides were extracted from the gel into 5% formic acid:acetronitrile (1:1), and a second extraction into 5% formic acid:acetonitrile (5:95). The extracts were pooled, the volume reduced by vacuum centrifugation, and the final volume was brought up to 10 microliters with 0.1% TFA. Contaminating salts and particulates were removed by binding the peptides to a C₁₈-ZipTip (Millipore, Mass.), washing with 0.1% TFA, and elution into 10 microliters of 0.1% TFA: acetonitrile (1:1). The peptides from the tryptic digests were analyzed by tandem liquid chromatography/mass spectrometry (LC-MS/MS). Liquid chromatography was performed using a Michrom Magic HPLC system with a constant pressure splitter to reduce the flow rate through the column to 400 nl/min. Peptides were separated by reversed phase chromatography, using Vydac C₁₈, 5 micrometer particle, 300 angstrom pore packing. A column of approximately 5 cm was packed into a 75 micrometer I.D. fused silica capillary (PicoFrit, New Objective Inc., Woburn Mass.). Peptides were separated using a linear gradient from 2-85% buffer B (Buffer A: 5% acetonitrile in water with 0.5% acetic acid and 0.005% TFA; Buffer B: 80% acetonitrile, 10% n-propanol, 10% water, with 0.5% acetic acid, 0.005% TFA). The LC effluent was electrosprayed directly into the sampling orifice of an LCQ DECA spectrometer (Thermo Finnigan, Calif.) using an adaption of the microscale electrospray interface[45]. The LCQ DECA was operated to collect MS/MS spectra in a data dependent manner, with up to four of the most intense ions that exceeded a pre-set threshold being subjected to fragmentation and analysis. The MS/MS data generated was analyzed and matches to protein sequences in the NCBI non-redundant database (mammalian subset) were determined using both SEQUEST [46] and MASCOT [47] programs.

Sequence identification was based on the Mowse score [48] (10×log(P), where P is the probability that the observed match found by the Mascot software is a random event). Protein scores greater than 60 were significant at p<0.05. In each case, the predicted Mᵣ and pI of the identification matched the observed Mᵣ and pI values within ±5%.

Computer analysis: Image quantitation, spot alignment, and molecular weight estimation were done using the image analysis program tnimage[49]. available at (http://) entropy.brni-jhu.org/tnimage.html).

### REFERENCES

1. S. E. Benashski and S. M. King. Investigation of protein-protein interactions within flagellar dynein using homobifunctional and zero-length crosslinking reagents. Methods, 22:365-371 (2000).
2. C. T. Rollins, et al. A ligand-reversible dimerization system for controlling protein-protein interactions. Proc. Natl. Acad. Sci. USA, 97:7096-7101 (2000).
3. S. A. McMahan and R. R. Burgess. Use of aryl azide cross-linkers to investigate protein-protein interactions: An optimization of important conditions as applied to Eschericia coli RNA polymerase and localization of a sigma 70-alpha cross-link to the C-terminal region of alpha. Biochemistry, 33:12092-12099 (1994).
4. S. Kiessig, J. Reissmann, C. Rascher, G. Kullertz, A. Fischer, and F. Thunecke. Application of a green fluorescent fusion protein to study protein-protein interactions by electrophoretic methods. Electrophoresis, 22:1428-1435 (2001).
5. S. H. Park and R. T. Raines. Green fluorescent protein chimeras to probe protein-protein interactions. Methods Enzymol, 328:251-261 (2000).
6. G. P. Smith. Filamentous fusion phage: novel expression vectors that display cloned antigens on the virion surface. Science, 228:1315-1317 (1985).
7. S Rossenu, D. Dewitte, J. Vandekerckhove, and C. Ampe. A phage display technique for a fast, sensitive, and systematic investigation of protein-protein interactions. J. Protein Chem., 16:499-503 (1997).
8. S. Fields and O. Song. A novel genetic system to detect protein-protein interactions. Nature, 340:245-247 (1989).
9. G. MacBeath and S. L. Schreiber. Printing proteins as microarrays for high-throughput function determination. Science, 289:1760-1763 (2000).
10. M. N. Kronick and W. A. Little. A new immunoassay based on fluorescence excitation by internal reflection spectroscopy. J. Immunol. Methods, 8:235-240 (1995).
11. J. D. Andrade, D. E. Van Wagenen, D. E. Gregonis, K. Newby, and J. N Lin. Remote fiber optic biosensors based on evanescent-excited fluoroimmunoassay: concepts and progress. IEEE Trans. Elec. Dev., ED-32:1175-1179 (1985).
12. S. Beeckmans. Chromatographic methods to study protein-protein interactions. Methods, 19:278-305 (1999).
13. A. K. Kenworthy. Imaging protein-protein interactions using fluorescence resonance energy transfer microscopy. Methods, 24:289-296 (2001).
14. N. Mochizuki, S. Yamashita, K. Kurokawa, Y. Ohba, T. Nagai, A. Miyawaki, and M. Matsuda. Spatio-temporal images of growth-factor-induced activation of ras and rap1. Nature, 411:1065-1068 (2001).
15. A. Kumar and M. Snyder, Nature 415:123-124 (2002).
16. A.-C. Gavin et al. Functional organization of the yeast proteome by systematic analysis of protein complexes. Nature 415:141-147 (2002).
17. Y. Ho et al. Systematic identification of protein complexes in Saccharomyces cerevisiae by mass spectrometry. Nature 415:180-183 (2002).
18. J. Rappsilber, S. Siniossoglou, E. Hurt, M. Mann. A generic strategy to analyze the spatial organization of multi-protein complexes by cross-linking and mass spectrometry. Anal. Chem., 72:267-275 (2000).
19. P. Uetz, et al. A comprehensive analysis of protein-protein interactions in saccharomyces cerevisiae. Nature, 403:623-627 (2000).
20. T. Ito, T. Chiba, R. Ozawa, M. Yoshida, M. Hattori, and Y. Sakaki. A comprehensive two-hybrid analysis to explore the yeast protein interactome. Proc. Natl. Acad. Sci. USA., 98(8):4569-4574 (2001).
21. C. Paweletz et al. Reverse phase protein microarrays which capture disease progression show activation of pro-survival pathways at the cancer invasion front. Oncogene 20(16):1981-1989 (2001).
22. P. Wagner et al. Arrays of protein-capture agents and methods of use thereof. U.S. Pat. No. 6,329,209 (2001).
23. T. Nelson, P. Backlund, Jr., A. Yergey, and D. Alkon. Isolation of Protein Subpopulations Undergoing Protein-Protein Interactions. Mol. Cell. Proteomics, 1:253-259 (2002).
24. R. Morris. Developments of a water-maze procedure for studying spatial learning in the rat. J. Neurosci. Methods, 11(1):47-60 (1984).
25. R. Brandeis, Y. Brandys, and S. Yehuda. The use of the morris water maze in the study of memory and learning. Intern. J. Neurosci., 48:29-69 (1989).
26. J. C. Tercero and Diaz-Maurino. T. Affinity chromatography of fibrinogen on lens culinaris agglutinin immobilized on CNBr-activated sepharose: study of the active groups involved in nonspecific adsorption. Anal Biochem, 174(1):128-136 (1988).
27. S. Beeckmans, ref. 12.
28. M. P. Washburn, D. Wolters, J. R. Yates III. Large-scale analysis of the yeast proteome by multidimensional protein identification technology. Nature Biotechnology 19:242-247 (2001).
29. L. Li et al. High Throughput Peptide Identification from Protein Digests Using Data-Dependent Multiplexed Tandem FTICR Mass Spectrometry Coupled with Capillary Liquid Chromatography. Anal. Chem. 73:3312-3322 (2001).
30. M. J. Hubbard and N. J. McHuhh. Mitochondrial ATP synthase Fl-beta subunit is a calcium-binding protein. FEBS Lett., 391(3):323-329 (1996).
31. N. Arakaki, Y. Ueyama, M. Hirose, T. Himeda, H. Shibata, S. Futaki, K. Kitagawa, and T. Higuti. Stoichiometry of subunit e in rat liver mitochondrial H(+)-ATP synthase and membrane topology of its putative Ca(2+)-dependent regulatory region. Biochim. Biophys. Acta, 1504(2-3):220-228 (2001).
32. E. W. Yamada and N. J. Huzel. The calcium-binding ATPase inhibitor protein from bovine heart mitochondria. purification and properties. J. Biol. Chem., 263(23):11498-11503 (1988).
33. B. W. Schafer and C. W. Heizmann. The S100 family of EF-hand calcium-binding proteins. Trends in Biochem., 21:134-139 (1996).
34. R. Bosser, M. Faura, J. Serratosa, J. Renau-Piqueras, M. Pruschy, and O. Bachs. Phosphorylation of rat liver heterogeneous nuclear ribonucleoproteins a2 and c can be modulated by calmodulin. Mol. Cell. Biol., 15(2):661-670 (1995).
35. S. Treves, E. Scutari, M. Robert, S. Groh, M. Ottolia, G. Prestipino, M. Ronjat, and F. Zorzato. Interaction of S100A1 with the Ca2+ release channel (ryanodine receptor) of skeletal muscle. Biochemistry, 36(38):11496-11503 (1997).
36. N. L. Golitsina, J. Kordowska, C. L. Wang, and S. S. Lehrer. Ca2+-dependent binding of calcyclin to muscle tropomyosin. Biochem Biophys Res Commun, 220(2):360-365 (1996).
37. N. K. Noren, C. M. Niessen, B. M. Gumbiner, and K. Burridge. Cadherin engagement regulates rho family GTPases. J. Biol. Chem., 276(36):33305-33308 (2001).
38. T. Kaneko, M. Amano, A. Maeda, H. Goto, K. Takahashi, M. Ito, and K. Kaibuchi. Identification of calponin as a novel substrate of rho-kinase. Biochem. Biophys. Res. Commun., 273(1):110-116 (2000).
39. H. Weinstein and E. L. Mehler. Ca2+ binding and structural dynamics in the functions of calmodulin. Ann. Rev. Physiol., 56:213-236 (1994).
40. E. Friedrichs, R. A. Farley, and H. J. Meiselman. Influence of calcium permeabilization and membrane-attached hemoglobin on erythrocyte deformability. Am. J. Hematol., 41(3):170-177 (1992).
41. M. Sudol, Sliwa K, and Russo T. Functions of WW domains in the nucleus. FEBS Lett., 490(3):190-195 (2001).
42. J. Q. Trojanowski and V. M. Lee. Fatal attractions of proteins. a comprehensive hypothetical mechanism underlying alzheimer's disease and other neurodegenerative disorders. Ann. N.Y. Acad. Sci., 924:62-67 (2000).
43. M. M. Bradford. A rapid and sensitive method for the quantitation of microgram quantities of protein utilizing the principle of protein-dye binding. Anal. Biochem., 72:248-254 (1976).
44. Association of Biomolecular Resource Facilities. Representative “In-Gel” Digestion Protocol for Proteins in SDS PAGE Gel. (1977). (http://) www.abrf org/ABRF/ResearchCommittees/intprotseqrescomm.html
45. M. T. Davis, D. C. Stahl, S. A. Hefta, and T. D. Lee. A microscale electrospray interface for on-line, capillary liquid chromatography/tandem mass spectrometry of complex peptide mixtures. Anal. Chem., 24:45494556 (1995).
46. J. K. Eng, A. L. McCormack, and J. R. III. Yates. An approach to correlate tandem mass spectral data of peptides with amino acid sequences in a protein database. J. Amer. Soc. Mass Spec., 5:976-989 (1994).
47. D. N. Perkins, D. J. Pappin, D. M. Creasy, and J. S. Cottrell. Probability-based protein identification by searching sequence databases using Mass spectrometry data. Electrophoresis, 18:3551-3567 (1999).
48. D. Pappin, P. Højrup, and A. Bleasby. Rapid identification of proteins by peptide-mass fingerprinting. Curr. Biol. 3(6):327-332 (1993)
49. The tnimage program is available at (http://) entropy.brni-jhu.org/tnimage.html.

We claim:

1. A method of isolating, from a mixture of proteins, a subpopulation consisting essentially of proteins that engage in protein-protein interactions, comprising:
(a) contacting the mixture with a chemically reactive support under conditions that permit
(i) covalent binding of proteins to the support, and
(ii) protein-protein interactions;
(b) permitting proteins in the mixture to become covalently bound to the support;
(c) separation of the support from any proteins not bound thereto;
(d) subjecting the support to conditions that disrupt protein-protein interactions; and
(e) separating the support from any proteins not bound thereto.

2. The method of claim 1, wherein the chemically reactive support comprises chemically reactive moieties selected from the group consisting of: cyanate groups, isocyanate groups, isothiocyanate groups, activated carboxyl groups, activated sulfonyl groups, aldehyde groups, epoxide groups, and thiol groups.

3. The method of claim 2, wherein the chemically reactive support comprises cyanate groups.

4. The method according to any one of claims 1-3, wherein the support comprises an optionally cross-linked polymer or gel.

5. The method of claim 4, wherein the support comprises a material selected from the group consisting of polystyrene, agar, agarose, polyacrylamide, dextran, hydroxylated vinyl polymers, and carboxylated vinyl polymers.

6. The method of claim 5, wherein the support comprises agarose.

7. In a method for analyzing a mixture of proteins, which comprises contacting said mixture with an array of immobilized proteins, the improvement which consists of isolating, from said mixture of proteins, a subpopulation consisting essentially of proteins that engage in protein-protein interactions, and subsequently contacting said subpopulation with said array, wherein the method of isolating the subpopulation comprises:
(a) contacting the mixture with a chemically reactive support under conditions that permit
(i) covalent binding of proteins to the support, and
(ii) protein-protein interactions;
(b) permitting proteins in the mixture to become covalently bound to the support;
(c) separation of the support from any proteins not bound thereto;
(d) subjecting the support to conditions that disrupt protein-protein interactions; and
(e) separating the support from any proteins not bound thereto.
